(19) United States
(12) Patent Application Publication
(10) Pub. No.: US 2024/0268979 A1
(43) Pub. Date: Aug. 15, 2024

(54) BIFURCATED STENTS AND DELIVERY SYSTEMS FOR DEPLOYMENT THEREOF

(71) Applicant: M&L Healthcare Investments Pte. Ltd., SG (SG)

(72) Inventors: Roberto FERRARESI, Milan (IT); Wei Cheong Steven KUM, Singapore (SG)

(73) Assignee: M&L Healthcare Investments Pte. Ltd., SG (SG)

(21) Appl. No.: 18/419,506

(22) Filed: Jan. 22, 2024

Related U.S. Application Data
(60) Provisional application No. 63/484,933, filed on Feb. 14, 2023.

(51) Int. Cl. A61F 2/954; A61F 2/966

(52) U.S. Cl. CPC A61F 2/954; A61F 2/9662

(57) ABSTRACT

Systems and methods for implanting a bifurcated stent comprising a main portion and a side portion within a blood vessel and an adjoining anastomosis are provided. The system includes a Y-shaped inner shaft having a first portion for supporting the main portion of the bifurcated stent and a second portion for supporting the side portion of the bifurcated stent, a first outer shaft for receiving the main portion of the bifurcated stent in a collapsed delivery state over the first portion of the inner shaft, and a second outer shaft for receiving the side portion of the bifurcated stent in a collapsed delivery state over the second portion of the inner shaft. The first and second outer shafts may be retracted relative to the bifurcated stent to deploy the bifurcated stent within the target blood vessel and adjoining anastomosis.

### CROSS-REFERENCE TO RELATED APPLICATION

This application claims priority to U.S. Provisional Patent Appl. No. 63/484,933, filed Feb. 14, 2023, the entire contents of which are incorporated herein by reference.

### FIELD OF USE

The present disclosure is directed to systems and methods for delivering a bifurcated stent to a blood vessel and adjoining anastomosis.

### BACKGROUND

Many people suffering from peripheral artery disease suffer from insufficient blood flow to peripheral regions of their body. This may be caused by the narrowing or occlusion of one or more peripheral arteries, e.g., a superficial femoral artery, to their peripheral extremities, e.g., legs or arms. Such narrowing or occlusion of the vessels may reduce blood flow to one or more peripheral regions. Insufficient blood flow to the extremities of the body can lead to claudication, chronic limb threatening ischemia, gangrene, and/or amputation.

Previous attempts to restore normal blood flow through the vessel includes ablating the occlusion to clear the passageway through the vessel as described in U.S. Pat. No. 8,545,418 to Heuser, or to surgically bypass the occlusion. For example, U.S. Pat. No. 8,062,321 to Heuser describes systems and methods for creating a fistula between two adjacent blood vessels to allow blood to exit the occluded vessel upstream of the occlusion, flow into the adjacent vessel, and back in to the original vessel at a point downstream of the occlusion.

An issue arises in co-locating the openings in the two blood vessels and holding the vessel walls in place to ensure that a channel will be created between the vessels so that blood will flow from one vessel to the other. Another issue involves aiming and maintaining the position of the catheters inside the vessels. In particular, veins often have diameters much larger than arteries, making hitting a smaller artery from a larger vein difficult. Additionally, larger veins often allow a catheter too much freedom of movement inside the vein. Another issue is related to the type of the new created anastomosis, which is termino-terminal, completely deviating the blood flow from the target artery in the new channel and occluding the original blood flow inside the target artery, if present.

Therefore, it is desirable to provide an improved bifurcated stent, as well as effective systems and methods for implanting the bifurcated stent in a desired position within a blood vessel and adjoining anastomosis. For example, a latero-terminal anastomosis may be preferable as it can maintain the blood flow inside the target artery.

### SUMMARY

The present disclosure overcomes the drawbacks of previously-known systems and methods by providing a bifurcated stent, and systems and methods for implanting the bifurcated stent comprising a main portion and a side portion within a blood vessel and an adjoining anastomosis. The system may include an inner shaft comprising a first portion configured to support the main portion of the bifurcated stent and a second portion configured to support the side portion of the bifurcated stent, a first outer shaft comprising a lumen sized and shaped to receive the main portion of the bifurcated stent in a collapsed delivery state over the first portion of the inner shaft, and a slot extending longitudinally from a proximal end of the first outer shaft, the slot sized and shaped to accommodate the side portion of the bifurcated stent, and a second outer shaft comprising a lumen sized and shaped to receive the side portion of the bifurcated stent in a collapsed delivery state over the second portion of the inner shaft. The first outer shaft may be configured to be retracted distally relative to the bifurcated stent to transition the main portion of the bifurcated stent from its collapsed delivery state to an expanded deployed state within the blood vessel. In addition, the second outer shaft may be configured to be retracted proximally relative to the bifurcated stent to transition the side portion of the bifurcated stent from its collapsed delivery state to an expanded deployed state within the anastomosis.

The second portion of the inner shaft may extend from a side of the first portion of the inner shaft. The inner shaft may have a guidewire lumen extending entirely through the first portion of the inner shaft. Additionally, the inner shaft may have a guidewire lumen extending through at least a portion of the first portion of the inner shaft and entirely through the second portion of the inner shaft. The inner shaft may have a stiffness configured to facilitate alignment of the main portion of the bifurcated stent within the blood vessel and the side portion of the bifurcated stent within the anastomosis. The first and second outer shafts may be sized and shaped for advancement through the anastomosis to the blood vessel. A distal end of the second outer shaft may include one or more flaps configured to engage with the main portion of the bifurcated stent within the first outer shaft. Accordingly, the slot of the first outer shaft may be sized and shaped to permit the second outer shaft to extend therethrough while preventing the one or more flaps from being pulled through the slot when the one or more flaps are disposed over the main portion of the bifurcated stent within the first outer shaft.

In addition, the system further may include a handle configured to be removably coupled to at least one of a distal region of the first outer shaft or a proximal region of the second outer shaft. For example, the handle may have a lumen having a first threaded surface, and the at least one of the distal region of the first outer shaft or the proximal region of the second outer shaft may have a corresponding threaded surface configured to be removably coupled to the first threaded surface. In some embodiments, the handle may include an engagement tube having a lumen sized and shaped to receive at least one of the first portion of the inner shaft or the second portion of the inner shaft. The engagement tube may be actuated to transition between an unlocked state where the lumen slidably receives the at least one of the first portion of the inner shaft or the second portion of the inner shaft and a locked state where the lumen engages and secures the at least one of the first portion of the inner shaft or the second portion of the inner shaft to the engagement tube. The engagement tube may include a pair of locking portions, and the handle may include a slider having a tapered portion defining a slot extending within the slider. Accordingly, the slider may be actuated to transitionally move along the pair of locking portions between an unlocked position where the slider is not engaged with the pair of locking portions and a locked position where the pair of locking portions is received within the slot and moved toward each other via the tapered portion to decrease an area of the lumen of the engagement tube to thereby secure the at least one of the first portion of the inner shaft or the second portion of the inner shaft to the engagement tube.

Moreover, the handle may include an actuator configured to be actuated to translationally move the at least one of the distal region of the first outer shaft or the proximal region of the second outer shaft relative to the at least one of the first portion of the inner shaft or the second portion of the inner shaft, respectively, when the at least one of the distal region of the first outer shaft or the proximal region of the second outer shaft is removably coupled to the handle and the at least one of the first portion of the inner shaft or the second portion of the inner shaft is secured to the engagement tube. For example, the actuator may include a pinion, and the handle further may include a rack configured to moveably engage with the pinion to translationally move the at least one of the distal region of the first outer shaft or the proximal region of the second outer shaft relative to the at least one of the first portion of the inner shaft or the second portion of the inner shaft, respectively.

In addition, a distal region of the first outer shaft may be configured to be removably coupled to a handle. For example, an outer surface of the distal region of the first outer shaft may have a first threaded surface, and the handle may have a lumen having a second threaded surface configured to be removably coupled to the first threaded surface. In addition, a proximal region of the second outer shaft may be configured to be removably coupled to a handle. For example, an outer surface of the proximal region of the second outer shaft may have a first threaded surface, and the handle may have a lumen having a second threaded surface configured to be removably coupled to the first threaded surface.

When the main portion of the bifurcated stent is disposed within the lumen of the first outer shaft in its collapsed delivery state, the side portion of the bifurcated stent may extend through the slot of the first outer shaft. Accordingly, as the first outer shaft is retracted distally relative to the bifurcated stent, the side portion of the bifurcated stent may be guided along the slot of the first outer shaft. The system further may include the bifurcated stent comprising the main portion and the side portion. The bifurcated stent may be configured to transition between a collapsed delivery state and an expanded deployed state. Moreover, a portion of the bifurcated stent adjacent to where the main portion is coupled to the side portion may have a stiffness greater than other portions of the bifurcated stent.

In addition, the system may include a first sheath sized and shaped for advancement through the anastomosis to the blood vessel, the first sheath comprising a lumen sized and shaped to receive the first and second outer shafts therethrough. A distal end of the sheath may have a tapered profile. Moreover, the first sheath may be sized and shaped for advancement through the anastomosis to the blood vessel via a homolateral access point of the blood vessel. Additionally, the system may include a second sheath sized and shaped for advancement through the blood vessel via a remote access point, e.g., a contralateral access point, of the blood vessel, the second sheath comprising a lumen sized and shaped to receive the first outer shaft therethrough.

In accordance with another aspect of the present disclosure, a method for implanting the bifurcated stent comprising the main portion and the side portion within the blood vessel and an adjoining anastomosis is provided. The method may include inserting a first sheath within the blood vessel via a homolateral access point; inserting a second sheath within the blood vessel via a remote access point; inserting a guidewire through the first sheath and the second sheath; advancing an inner shaft over the guidewire through the anastomosis to the blood vessel, the inner shaft supporting the bifurcated stent in a collapsed delivery state, the main portion of the bifurcated stent disposed within a first outer shaft in the collapsed delivery state and the side portion of the bifurcated stent disposed within a second outer shaft in the collapsed delivery state, the first outer shaft comprising a longitudinally extending slot sized and shaped to accommodate the side portion of the bifurcated stent; positioning the first outer shaft within the blood vessel and the second outer shaft within the anastomosis; retracting the first outer shaft distally relative to the bifurcated stent via the remote access point to transition the main portion of the bifurcated stent from the collapsed delivery state to an expanded deployed state within the blood vessel, such that the side portion of the bifurcated stent is guided along the slot of the first outer shaft; and retracting the second outer shaft proximally relative to the bifurcated stent via the homolateral access point to transition the side portion of the bifurcated stent from its collapsed delivery state to an expanded deployed state within the anastomosis.

A first portion of the inner shaft may support the main portion of the bifurcated stent in the collapsed delivery state within the first outer shaft, and a second portion of the inner shaft extending from the first portion of the inner shaft may support the side portion of the bifurcated stent in the collapsed delivery state within the second outer shaft. Advancing the inner shaft over the guidewire may comprise advancing the inner shaft over the guidewire via a guidewire lumen extending through at least a portion of the first portion of the inner shaft and through the second portion of the inner shaft. The method further may include inserting a second guidewire through a second guidewire lumen extending entirely through the first portion of the inner shaft, such that the second guidewire is disposed through the remote access point within the blood vessel to facilitate deployment of the bifurcated stent.

Moreover, advancing the inner shaft over the guidewire may comprise advancing the inner shaft and the first outer shaft through the first sheath and the second sheath such that distal ends of the inner shaft and the first outer shaft extend through the remote access point. The method further may include coupling a handle to the distal end of the first outer shaft, such that retracting the first outer shaft distally relative to the bifurcated stent comprises retracting the first outer shaft via the handle. In addition, the method may include coupling a handle to a proximal end of the second outer shaft, such that retracting the second outer shaft proximally relative to the bifurcated stent comprises retracting the second outer shaft via the handle. Positioning the first outer shaft within the blood vessel and the second outer shaft within the anastomosis may include visualizing a distal end of the first sheath via markers disposed on the distal end of the first sheath. The method further may include retracting the inner shaft distally relative to the bifurcated stent via the remote access point to remove the inner shaft from the blood vessel.

### BRIEF DESCRIPTION OF THE DRAWINGS

FIG. 1 illustrates an exemplary bifurcated stent constructed in accordance with the principles of the present disclosure.

FIG. 2 illustrates an exemplary delivery system for implanting a bifurcated stent in accordance with the principles of the present disclosure.

FIGS. 3A to 3D illustrate an exemplary inner shaft of the delivery system of FIG. 2.

FIGS. 4A to 4D illustrate an exemplary outer shaft of the delivery system of FIG. 2.

FIG. 4E illustrates an alternative exemplary side outer shaft constructed in accordance with the principles of the present disclosure.

FIGS. 5A and 5B illustrate an exemplary removable handle for use with the delivery system of FIG. 2.

FIGS. 5C to 5E illustrate components of the removable handle of FIGS. 5A and 5B.

FIGS. 5F and 5G illustrate locking of the removable handle to the inner shaft of the delivery system of FIG. 2.

FIGS. 6A and 6B illustrate an exemplary sheath for delivering the delivering system of FIG. 2.

FIG. 7 is a flow chart illustrating exemplary method steps for implanting a bifurcated stent using the delivery system of FIG. 2.

FIGS. 8A to 8D illustrate exemplary method steps for disposing the bifurcated stent on the inner shaft and within the outer shaft in a collapsed delivery state in accordance with the principles of the present disclosure.

FIG. 8E is a cross-sectional view of the delivery system of FIG. 8D.

FIGS. 9A to 9P illustrate exemplary method steps for implanting a bifurcated stent using the delivery system of FIG. 2.

FIGS. 10A and 10B illustrate an alternative exemplary removable handle constructed in accordance with the principles of the present disclosure.

FIGS. 11A and 11B illustrate an alternative exemplary outer shaft assembly constructed in accordance with the principles of the present disclosure.

FIG. 11C illustrates another alternative exemplary outer shaft assembly constructed in accordance with the principles of the present disclosure.

### DETAILED DESCRIPTION

In view of the foregoing, it would be desirable to provide bifurcated stent, e.g., for extravascularly bypassing an occlusion within a patient's blood vessel, as well as systems and methods for implanting the bifurcated state within a blood vessel and adjoining anastomosis. For example, the bifurcated stent may direct blood flow within blood vessel out of the vessel, and back to the blood vessel at a location downstream of the occlusion, thereby bypassing the occlusion within the blood vessel.

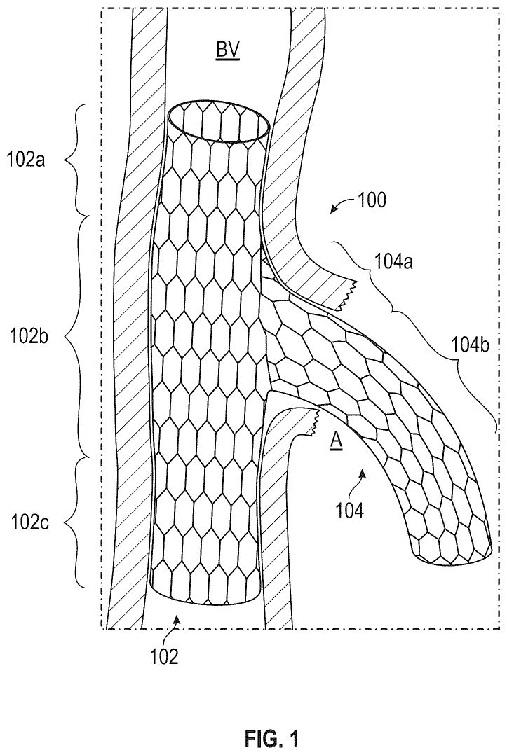

Referring now to FIG. 1, an exemplary bifurcated stent for implantation within a blood vessel and adjoining anastomosis is provided. As shown in FIG. 1, bifurcated stent 100 may include main portion 102 sized and shaped to be deployed within a target blood vessel, e.g., blood vessel BV, and side portion 104 extending from a side of main portion 102 and sized and shaped to be deployed within an anastomosis, e.g., anastomosis A, coupled to blood vessel BV. For example, main portion 102 may be implanted within blood vessel BV at a location upstream of an occlusion within blood vessel BV. Bifurcated stent 100 may be covered, e.g., by a flexible biocompatible material. Accordingly, main portion 102 may have a lumen fluidically coupled to the lumen of side portion 104, such that at least some blood flow through main portion 102 within blood vessel BV may be redirected through side portion 104 within anastomosis A. As will be understood by a person having ordinary skill in the art, the bifurcated stent described herein may be implanted at other locations along the patient's vasculature, e.g., downstream of an occlusion within a blood vessel.

Bifurcated stent 100 may be formed of an elastic material, e.g., a flexible metal frame such as Nitinol or other flexible materials, such that bifurcated stent 100 may transition between a collapsed delivery state and an expanded deployed state. Bifurcated stent 100 may be biased toward the expanded deployed state. As shown in FIG. 1, main portion 102 may include upstream portion 102A, middle portion 102B, and downstream portion 102C, and side portion 104 may include upstream portion 104A and downstream portion 104B. Accordingly, upstream portion 104A of side portion 104 may be fluidically coupled to and extended from middle portion 102B of main portion 102, e.g., to form a Y-shaped bifurcated stent. Moreover, middle portion 102B and upstream portion 104A may have a stiffness that is higher than the stiffness of upstream portion 102A, downstream portion 102C, and downstream portion 104B, to thereby provide sufficient radial force to blood vessel BV and anastomosis A adjacent to where anastomosis A is coupled to blood vessel BV, e.g., via a carina created in blood vessel BV, while maintaining sufficient flexibility along upstream portion 102A, downstream portion 102C, and downstream portion 104B to adapt to the surrounding vasculature and patient anatomy.

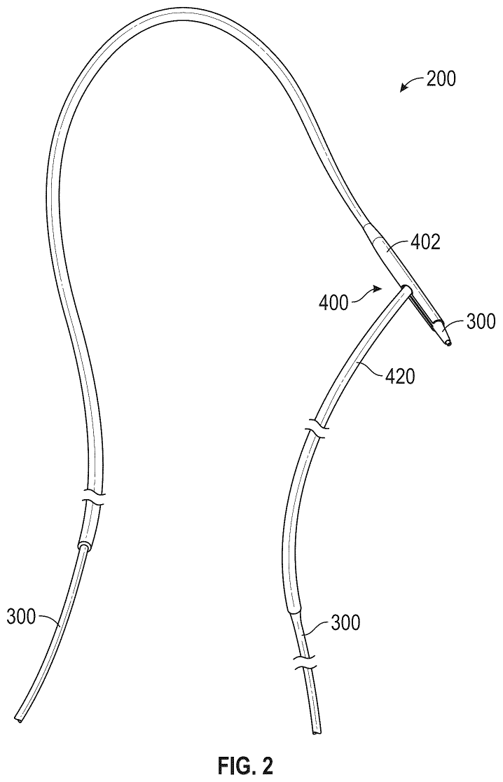

Referring now to FIG. 2, an exemplary delivery system for implanting a bifurcated stent within a blood vessel and adjoining anastomosis is provided. System 200 may include inner shaft 300, e.g., a Y-shaped inner shaft, and outer shaft 400 comprising first outer shaft 402 and second outer shaft 420, such that the bifurcated stent may be positioned over inner shaft 300, and crimped to a collapsed delivery state over inner shaft 300, within outer shaft 400. For example, main portion 102 of bifurcated stent 100 may be disposed within first outer shaft 402 over a first portion of inner shaft 300, and side portion 104 of bifurcated stent 100 may be disposed within second outer shaft 420 over a second portion of inner shaft 300, as described in further detail below, to prepare system 200 for delivery to the target blood vessel. Accordingly, system 200 may be sized and shaped for advancement through an anastomosis to a target blood vessel for deployment of bifurcated stent 100.

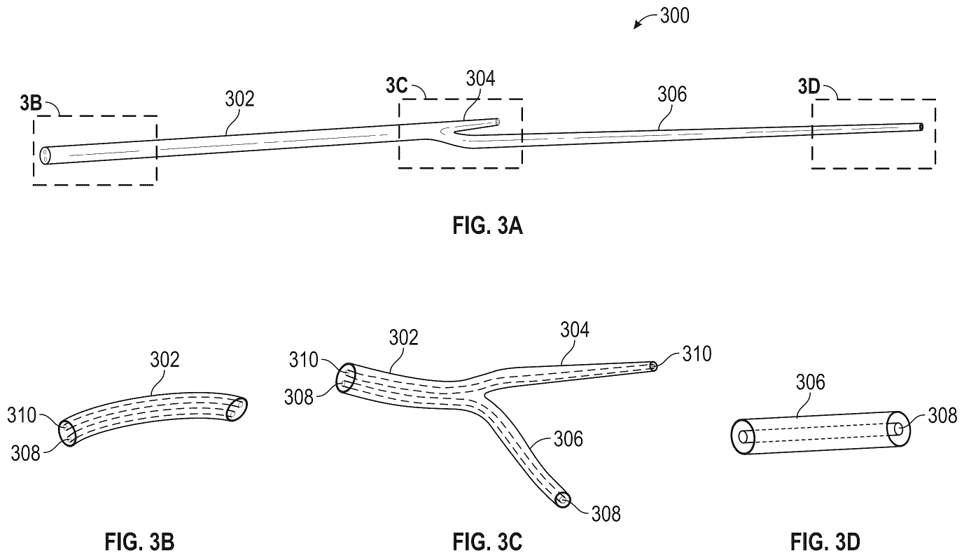

Referring now to FIGS. 3A to 3D, an exemplary inner shaft is provided. Inner shaft 300 may include a first main portion, e.g., distal main portion 302 and proximal main portion 304, sized and shaped to support main portion 102 of bifurcated stent 100 in the collapsed delivery state, and a second side portion 306 extending from the first main portion, e.g., between distal main portion 302 and proximal main portion 304, and sized and shaped to support side portion 104 of bifurcated stent 100 in the collapsed delivery state. As shown in FIG. 3A, side portion 306 extends from the main portion of inner shaft 300 at a point between distal main portion 302 and proximal main portion 304 to thereby form a Y-shape inner shaft. The position of side portion 306 relative to the main portion of inner shaft 300 may be selected to correspond with the geometry of bifurcated stent 100, e.g., the relative portion between side portion 104 and main portion 102 of bifurcated stent 100.

Accordingly, when bifurcated stent 100 is positioned at the target location within the blood vessel and adjoining anastomosis, distal main portion 302 and proximal main portion 304 may be disposed within the blood vessel, while side portion 306 extends within the anastomosis coupled to the blood vessel. Moreover, distal portion 302 may have a length selected to extend distally from the target location within the target blood vessel and through the blood vessel to outside the patient's body via a remote access point, and side portion 306 may have a length selected to extend proximally from the target location within the target blood vessel and through the adjoining anastomosis to outside the patient's body via a homolateral access point, as described in further detail below. In addition, inner shaft 300 may have a high stiffness sufficient to facilitate precise deployment of bifurcated stent 100, while providing flexibility in navigation through an anastomosis to the target blood vessel.

As shown in FIGS. 3B to 3D, inner shaft 300 may include first guidewire lumen 308 extending from the distal end of distal portion 302 through distal portion 302 and side portion 306 to the proximal end of side portion 306. Guidewire lumen 308 may be sized and shaped to receive a stiff guidewire, e.g., a 0.035″ guidewire, therethrough to guide inner shaft 300 through the anastomosis to the blood vessel over the guidewire. In addition, inner shaft 300 further may include second guidewire lumen 310 extending from the distal end of distal portion 302 through distal portion 302 and proximal portion 304 to the proximal end of proximal portion 304. Guidewire lumen 310 may be sized and shaped to receive another guidewire, e.g., a 0.018″ guidewire, therethrough to maintain alignment of system 200 within the blood vessel to facilitate proper deployment of bifurcated stent 100, as described in further detail below. Accordingly, distal portion 302 of inner shaft 300 may include two guidewire lumens, e.g., guidewire lumens 308 and 310, while proximal portion 304 of inner shaft 300 may include a single guidewire lumen, e.g., guidewire lumen 310, and side portion 306 of inner shaft 300 may include a single guidewire lumen, e.g., guidewire lumen 308.

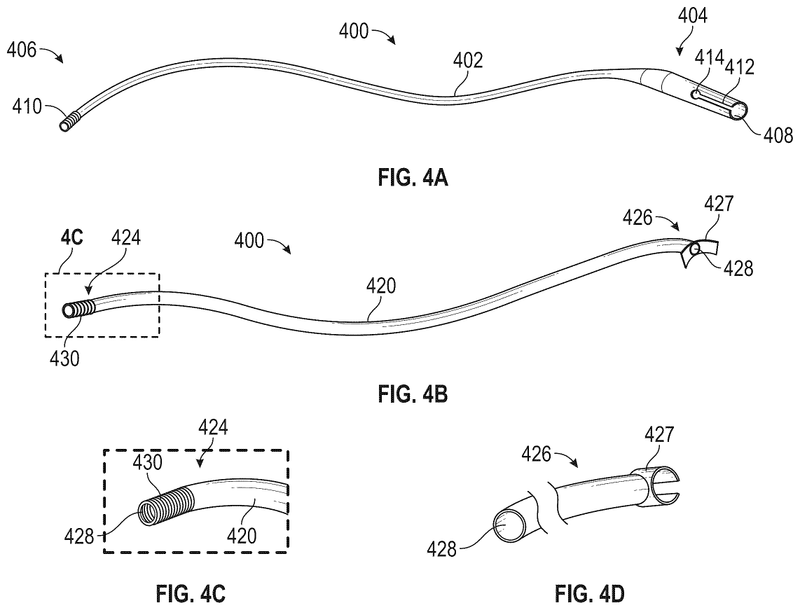

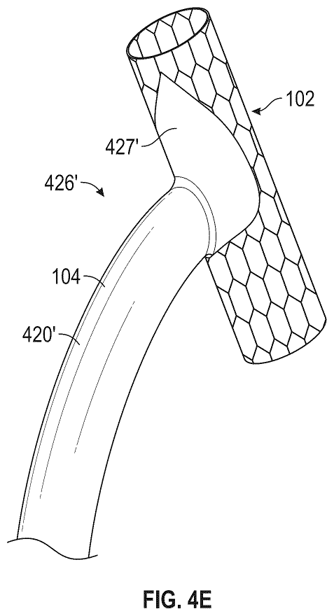

Referring now to FIGS. 4A to 4D, an exemplary outer shaft is provided. As described above, outer shaft 400 may include a main outer shaft, e.g., first outer shaft 402, and a side outer shaft, e.g., second outer shaft 420, each sized and shaped for advancement through an anastomosis to a target blood vessel. As shown in FIG. 4A, first outer shaft 402 of outer shaft 400 includes proximal region 404, distal region 406, and lumen 408 extending through first outer shaft 402 between proximal region 404 and distal region 406. Lumen 408 may be sized and shaped to receive main portion 102 of bifurcated stent 100 in its collapsed delivery state, e.g., when main portion 102 is crimped onto distal main portion 302 and proximal main portion 304 of inner shaft 300. In addition, first outer shaft 402 may have a length selected to extend distally from the target location within the target blood vessel and through the blood vessel to outside the patient's body via a contralateral access point.

As shown in FIG. 4A, proximal region 404 may include slot 412 extending longitudinally distally from the proximal end of proximal region 404. Slot 412 may be sized and shaped to accommodate side portion 104 of bifurcated stent 100, e.g., to guide side portion 104 therethrough as side portion 104 moves from a distal end of slot 412 proximally toward the proximal end of slot 412. As shown in FIG. 4A, the distal end of slot 412 may include opening 414 sized and shaped to accommodate side portion 104, e.g., when side portion 104 is in its collapsed delivery state within second outer shaft 420, as described in further detail below. For example, opening 414 may have a circumference that corresponds with the circumference of side portion 104 in its collapsed delivery state.

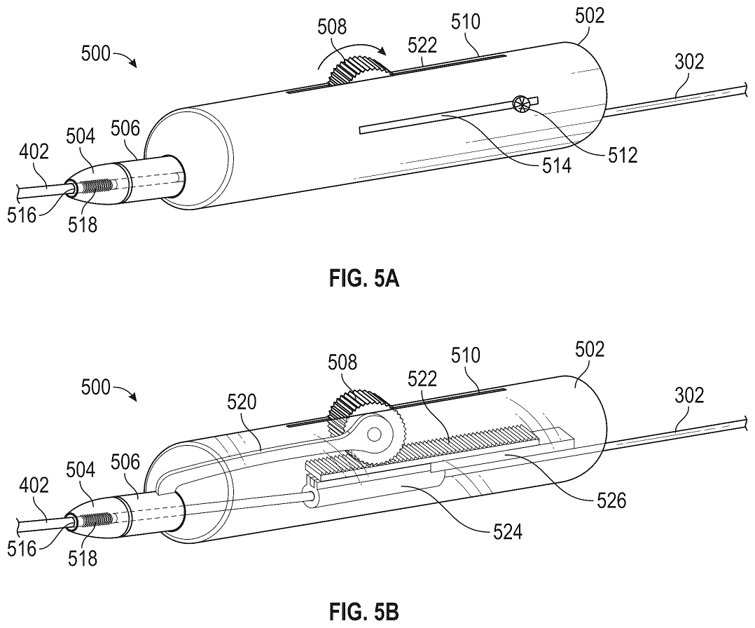

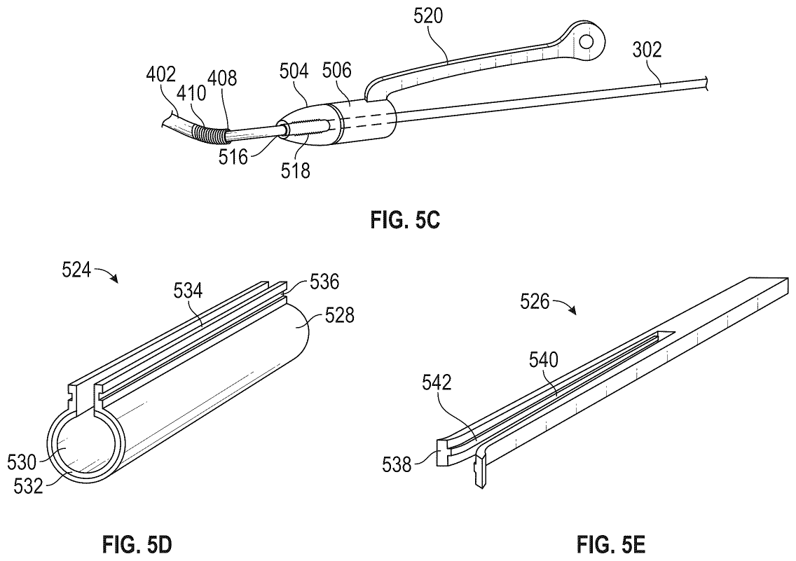

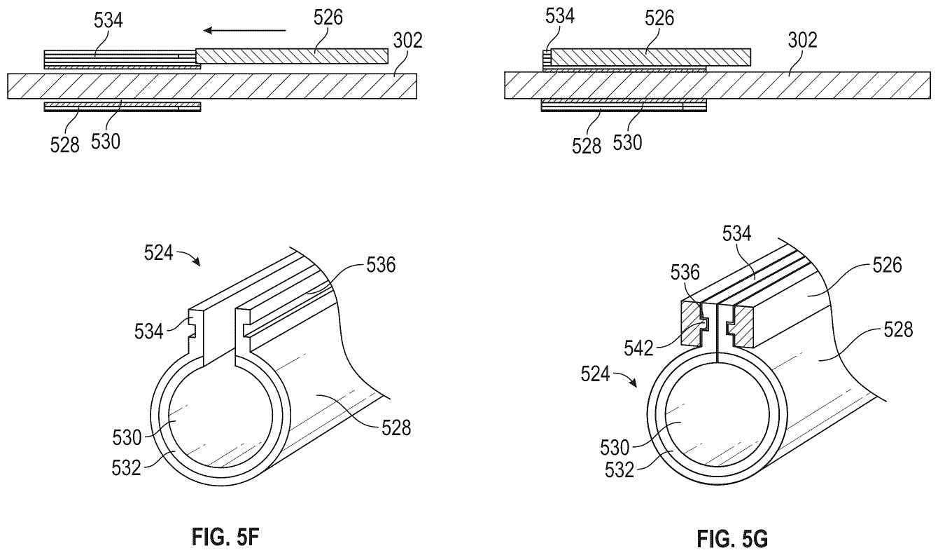

Moreover, slot 412 may have a smaller width, e.g., in a direction extending circumferentially along first outer shaft 402, than the width of opening 414. Accordingly, when side portion 104 is disposed within opening 414, slot 412 may facilitate securement of first outer shaft 402 by preventing incidental distal movement of first outer shaft 402 relative to bifurcated stent 100. However, when a sufficient force is applied to first outer shaft 402, first outer shaft 402 may be retracted distally relative to bifurcated stent 100, such that side portion 104 of bifurcated stent 100 moves from opening 414 proximally along slot 412, and main portion 102 of bifurcated stent 100 is exposed beyond the proximal end of first outer shaft 402 and transitions from its collapsed delivery state to its expanded deployed state. In addition, distal region 406 may be configured to be removably coupled to a handle, e.g., handle 500, as described in further detail below with regard to FIGS. 5A to 5E, which may be pulled by a user to facilitate retraction of first outer shaft 402 relative to bifurcated stent 100. For example, as shown in FIG. 4A, the outer surface of first outer shaft 402 at distal region 406 may include threaded surface 410, configured to releasably mate with the threaded surface of the handle, as described in further detail below.

As shown in FIG. 4B, second outer shaft 420 of outer shaft 400 includes proximal region 424, distal region 426, and lumen 428 extending through second outer shaft 420 between proximal region 424 and distal region 426. Lumen 428 may be sized and shaped to receive side portion 104 of bifurcated stent 100 in its collapsed delivery state, e.g., when side portion 104 is crimped onto side portion 306 of inner shaft 300. In addition, second outer shaft 420 may have a length selected to extend distally from the target location within the target blood vessel and through the anastomosis to outside the patient's body via a homolateral access point.

Moreover, a sufficient force may be applied to second outer shaft 420, e.g., via proximal region 424 to retract second outer shaft 420 proximally relative to bifurcated stent 100 and expose side portion 104 of bifurcated stent 100 beyond the distal end of second outer shaft 420, such that side portion 104 transitions from its collapsed delivery state to its expanded deployed state. In addition, proximal region 424 may be configured to be removably coupled to a handle, e.g., handle 500, as described in further detail below with regard to FIGS. 5A to 5E, which may be pulled by a user to facilitate retraction of second outer shaft 420 relative to bifurcated stent 100. For example, like distal region 406 of first outer shaft 402, as shown in FIG. 4C, the outer surface of second outer shaft 420 at proximal region 424 may include threaded surface 430, configured to releasably mate with the threaded surface of the handle.

Distal region 426 may include flaps 427, as shown in FIG. 4D. Flaps 427 may be configured to wrap around and engage at least a portion of main portion 102 of bifurcated stent 100, e.g., when main portion 102 is in its collapsed delivery state within first outer shaft 402. Accordingly, flaps 427 may be sandwiched between main portion 102 and first outer shaft 402 when first outer shaft 402 is disposed over flaps 427 and main portion 102. As shown in FIG. 4D, flaps 427 may include two flaps that extend from second outer shaft 420 and form a tubular shape so as to extend circumferentially around at least a portion of main portion 102. Accordingly, the distal ends of flaps 427 may have an edge that extends parallel to the longitudinal axis of main portion 102. Moreover, flaps 427 may be sized and shaped such that flaps 427 may not pass through opening 414 of first outer shaft 402 when first outer shaft 402 is disposed over main portion 102 and flaps 427 and second outer shaft 420 extends through opening 414, to thereby prevent inadvertent retraction of second outer shaft 420 relative to bifurcated stent 100 prior to retraction of first outer shaft 402 relative to bifurcated stent 100. Flaps 427 preferably are lubricious enough to slip off of main portion 102 during retraction of second outer shaft 420, while having a shape configured to facilitate removal of second outer shaft 420 from main portion 102 without displacing bifurcated stent 100.

As will be understood by a person having ordinary skill in the art, flaps 427 may have other shapes and sizes that prevent inadvertent retraction of second outer shaft 420 via opening 414 and facilitate removal of second outer shaft 420 from main portion 102 without displacing bifurcated stent 100. For example, as shown in FIG. 4E, flap 427′ may extend from distal region 426′ of second outer shaft 420′ in a direction parallel to the longitudinal axis of main portion 102 of bifurcated stent 100. Moreover, the distal end of flap 427′ may have a tapered profile to facilitate removal of flap 427′ from main portion 102.

Referring now to FIGS. 5A to 5G, an exemplary removable handle for coupling to outer shaft 400 and inner shaft 300 is provided. As described above, distal region 406 of first outer shaft 402 and proximal region 424 of second outer shaft 420 may include threaded surfaces 410 and 430, respectively, for removably coupled to handle 500. For example, as described in further detail below, when system 200 is delivered to and positioned at the target location within the patient's vasculature, e.g., main portion 102 is positioned within first outer shaft 402 within the target blood vessel and side portion 104 position within second outer shaft 420 within the adjoining anastomosis, distal region 406 of first outer shaft 402 may extend outside of the patient via the remote access point, and proximal region 424 of second outer shaft 420 may extend outside of the patient via the homolateral access point. A first handle may then be removably coupled to distal region 406 of first outer shaft 402 outside of the patient's body for maneuvering first outer shaft 402 relative to inner shaft 300, and a second handle may be removably coupled to proximal region 424 of second outer shaft 420 outside of the patient's body for maneuvering second outer shaft 420 relative to inner shaft 300.

As shown in FIGS. 5A and 5B, handle 500 may include handle body 502 configured to house internal components of handle 500, and sized and shaped to be held and controlled by a user. Handle 500 may include rotatable portion 504 rotatably coupled to stationary component 506, and lumen 516 extending through both rotatable portion 504 and stationary component 506. Lumen 516 may be sized and shaped to receive distal main portion 302 of inner shaft 300 and/or side portion 306 of inner shaft 300 therethrough. Moreover, the portion of lumen 516 extending through rotatable portion 504 may include threaded inner surface 518 configured to removably mate with threaded surface 410 of first outer shaft 402 and/or threaded surface 430 of second outer shaft 420. Accordingly, at least the portion of lumen 516 extending through rotatable portion 504 may be sized and shaped to receive the threaded surfaces of first outer shaft 402 and/or second outer shaft 420. For example, handle 500 may be advanced over distal main portion 302 and/or side portion 306, as shown in FIG. 5C (handle body 502 and other internal components of handle 500 omitted) until lumen 516 comes into contact with the threaded surfaces of first outer shaft 402 and/or second outer shaft 420. Rotatable portion 504 may then be rotated relative to stationary component 506 and first outer shaft 402 and/or second outer shaft 420 to receive the threaded surfaces of first outer shaft 402 and/or second outer shaft 420 within lumen 516 to thereby releasably secure handle 500 to outer shaft 400. As will be understood by a person having ordinary skill in the art, system 200 may include two separate handles for removably coupling to each of first outer shaft 402 and second outer shaft 420 during delivery of bifurcated stent 100, as described in further detail below.

As shown in FIGS. 5A and 5B, handle 500 further may include actuator 508 operatively coupled to stationary component 506 via bar 520, and configured to be actuated by a user to cause precise translational movement of stationary component 506 and rotatable component 504 relative to handle body 502. For example, as shown in FIG. 5B, when rotatable component 504 is removably coupled to first outer shaft 402, actuator 508 may be actuated to move stationary component 506, rotatable component 504, and first outer shaft 402 coupled thereto, relative to handle body 502 over distal main portion 302 of inner shaft 300 when distal main portion 302 is fixed relative to handle body 502, as described in further detail with regarding to FIGS. 5D to 5G. Referring again to FIG. 5B, actuator 508 may be rotatably coupled to a proximal end of bar 520, such that actuator 508 may be rotated relative to bar 520. Moreover, handle 500 may include a rack and pinion system for actuating actuator 508 and permitting translational movement of actuator 508 and stationary component 506 relative to handle body 502. For example, the outer edge of actuator 508 may have a geared surface, e.g., a pinion, sized and shaped to engage with rack 522 extending longitudinally within handle body 502 along the longitudinal axis of handle 500. Accordingly, as actuator 508 is rotated, the engagement between the geared outer edge of actuator 508 and rack 522 causes actuator 508, and accordingly stationary component 506, to translationally move relative to handle body 502, e.g., within slot 510 extending longitudinally along handle body 502.

In addition, handle 500 may include a mechanism for releasably engaging with inner shaft 300 to thereby secure inner shaft 300 relative to handle body 502. Fixing both inner shaft 300 and outer shaft 400 relative to handle 500, and accordingly to each other, is important for the safe advancement/maneuvering of bifurcated stent 100 in the collapsed delivery state to the desired position within the patient's vasculature to ensure proper deployment of bifurcated stent 100 at the target location. For example, as shown in FIGS. 5B, 5D, and 5E, handle 500 may include engagement tube 524 fixedly coupled to handle body 502, and configured to releasably engage with distal main portion 302 and/or side portion 306 of inner shaft 300. For example, as shown in FIG. 5D, engagement tube 524 may include engagement portion 528 having lumen 530 extending therethrough, such that lumen 530 is sized and shaped to receive distal main portion 302 and/or side portion 306 of inner shaft 300 therethrough. Engagement tube 524 is configured to transition between an unlocked state, where lumen 530 of engagement portion 528 may slidably receive inner shaft 300 therethrough, and a locked state where the diameter of lumen 530 decreases to thereby engage with and secure inner shaft 300 within engagement portion 528. Engagement tube 524 may be elastically biased toward the unlocked state. As shown in FIG. 5D, the inner surface of lumen 530 may include friction pad 532, e.g., a soft rubber surface, configured to engage with inner shaft 300 while providing frictional forces sufficient to secure inner shaft 300 to engagement tube 524 in the locked state.

In addition, engagement tube 524 may include a pair of locking portions 534 extending outwardly from engagement portion 528 along at least a portion of the length of engagement portion 528. As shown in FIG. 5D, engagement portion 528 may have a circular profile forming a tubular shape, and locking portions 534 may extend from the longitudinal edge of engagement portion 528. Moreover, locking portions 534 each may include groove 536 extending along the length of locking portions 534, and sized and shaped to releasably engage with slider 526 to transition engagement tube 524 between the unlocked and locked states. For example, as shown in FIG. 5E, handle 500 further may include slider 526 having tapered portion 538 and defining slot 540 having a predefined width extending longitudinally along at least a portion of the length of slider 526. Further, slider 526 may include a pair of protrusions 542 extending from tapered portion 538 through slot 540. Accordingly, upon translational movement of slider 526 distally relative to engagement tube 524, tapered portion 538 will facilitate receipt of locking portion 534 in the unlocked state, as shown in FIG. 5F, within slot 540 such that protrusions 542 slide along grooves 536 and the predefined width of slot 540 causes locking portions 534 to move toward each other to transition engagement tube 524 from the unlocked state to the locked state, as shown in FIG. 5G. As shown in FIG. 5A, slider 526 may be coupled to actuator 512, which may be actuated by a user to move within slot 514 extending longitudinally along handle body 502, and thereby move slider 526 from an unlocked position where slider 526 is not engaged with locking portion 534 and a locked position where locking portion 534 is disposed within slot 540.

Thus, upon coupling of rotatable portion 504 with outer shaft 400, actuator 526 may be actuated to transition engagement tube 524 from the unlocked position to the locked position where inner shaft 300 is secured to engagement tube 524, and thus, fixed relative to handle body 502. Actuator 508 may then be actuated to translationally move stationary component 506, rotatable component 504 and outer shaft 400 coupled thereto, relative to handle body 502, and accordingly, inner shaft 300, e.g., to thereby retract/remove outer shaft 400 from bifurcated stent 100 while inner shaft 300 remains stationary relative to the target blood vessel and adjoining anastomosis. By ensuring that inner shaft 300 remains stationary while outer shaft 400 is moved relative to inner shaft 300, accidental displacement of either or both shafts will be prevented.

To remove outer shaft 400 from the patient's body, actuator 526 may be actuated to transition engagement tube 524 from the locked position to the unlocked position to disengage inner shaft 300 from engagement tube 524. Handle 500 and outer shaft 400 coupled thereto may then be removed over inner shaft 300. In some embodiments, instead of engagement tube 524 and slider 526, handle 500 may include a Tuohy Borst valve configured to engage with and secure inner shaft 300 relative to handle body 502, such that outer shaft 400 may be precisely moved translationally relative to inner shaft 300 for safe deployment of bifurcated stent 100 at the proper anastomotic site while avoiding accidental displacement of either or both shafts and preventing erroneous deployment of bifurcated stent 100 at an improper location.

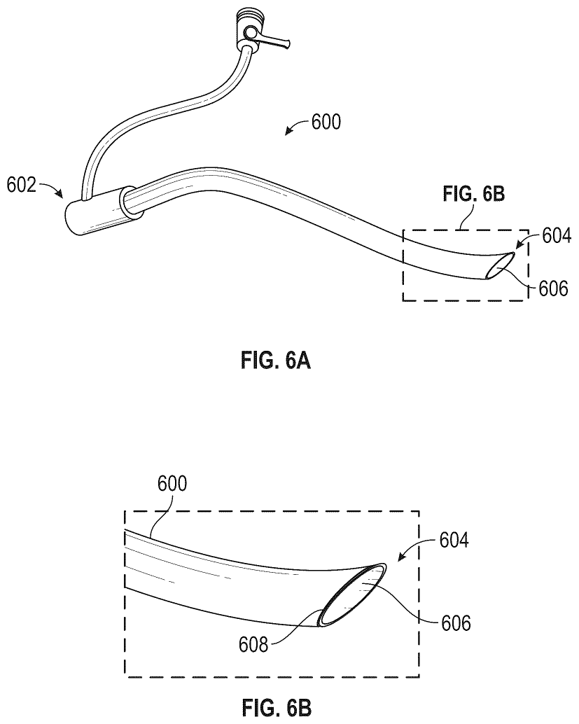

Referring now to FIGS. 6A and 6B, an exemplary dedicated delivery sheath is provided. System 200 may include at least one dedicated delivery sheath 600. As shown in FIG. 6A, sheath 600 may include inlet 602, outlet 604, and lumen 606 extending therethrough. Lumen 606 may be sized and shaped to receive outer shaft 400, e.g., both first outer shaft 402 and second outer shaft 420, and accordingly inner shaft 300 and bifurcated stent 100 disposed therebetween in its collapsed delivery state, therethrough. For example, sheath 600 may be a 14-24 Fr sheath. As shown in FIG. 6B, outlet 604 may have tapered profile, e.g., an oblique mouth, and marker 608, e.g., a radiopaque ring marker, disposed thereon to facilitate alignment of sheath 600 and bifurcated stent 100 during the delivery procedure. For example, the tapered angle of outlet 604 may be selected to correspond with the angle at which side portion 104 extends from main portion 102 of bifurcated stent 100 to facilitate alignment of main portion 102 within the target blood vessel and side portion 104 within the adjoining anastomosis. For example, outlet 604 may have a tapered angle of 45 degrees.

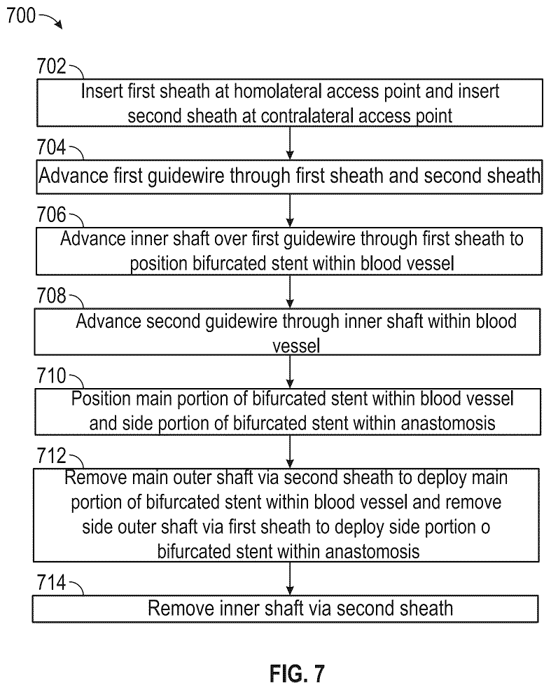

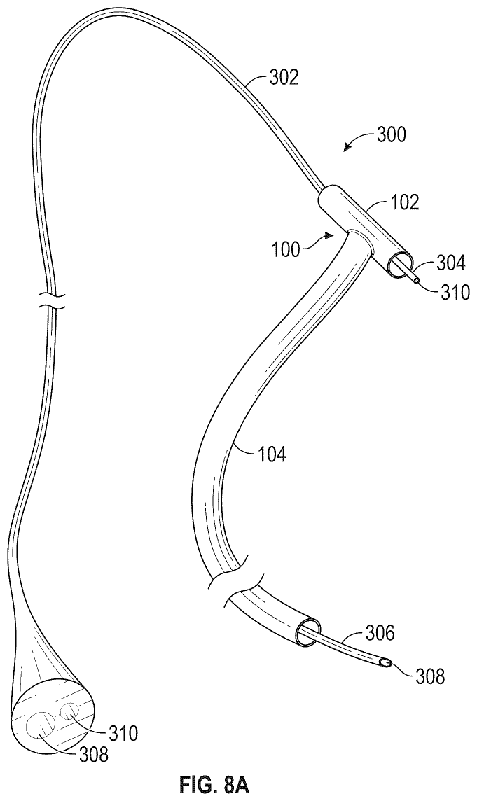

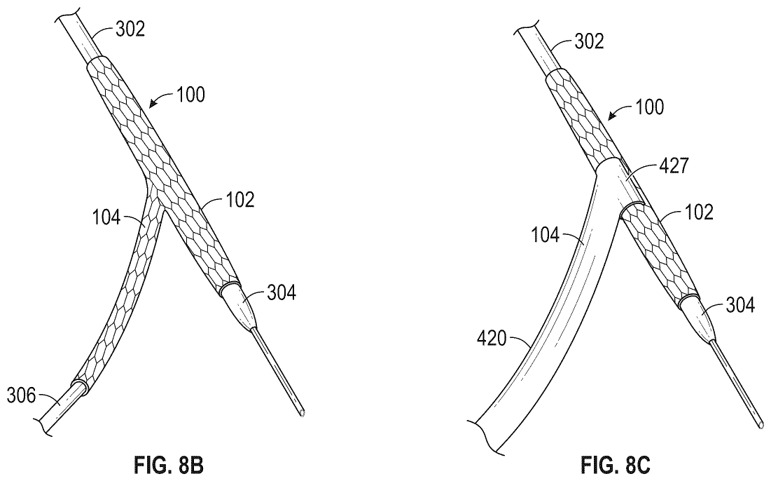

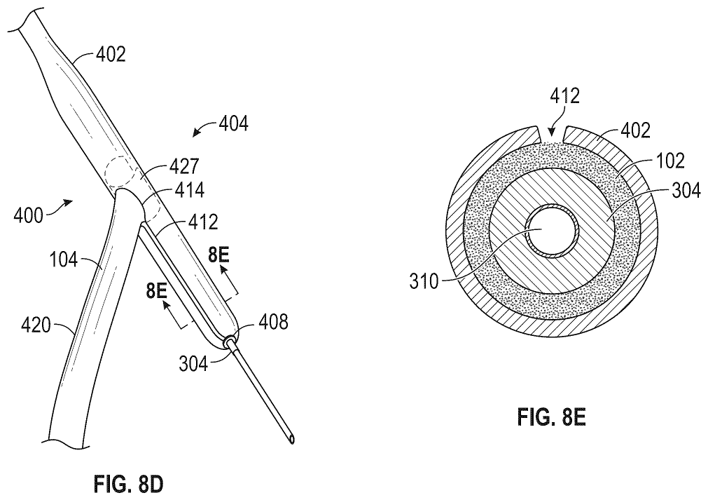

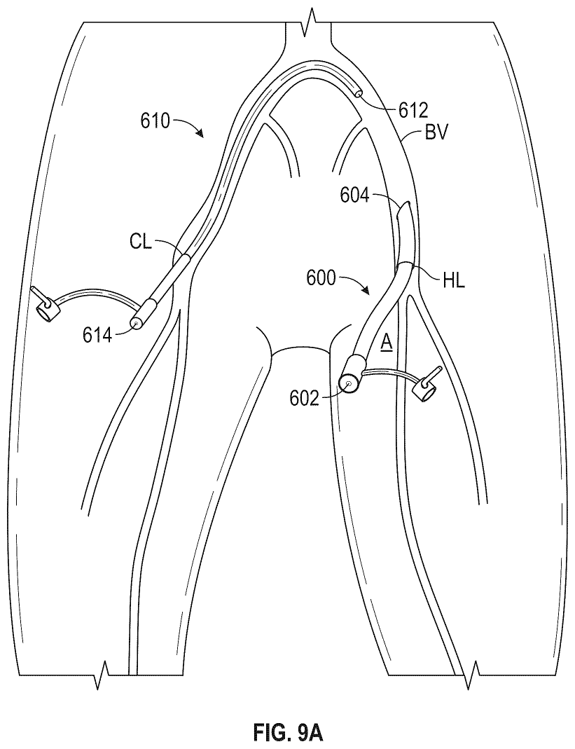

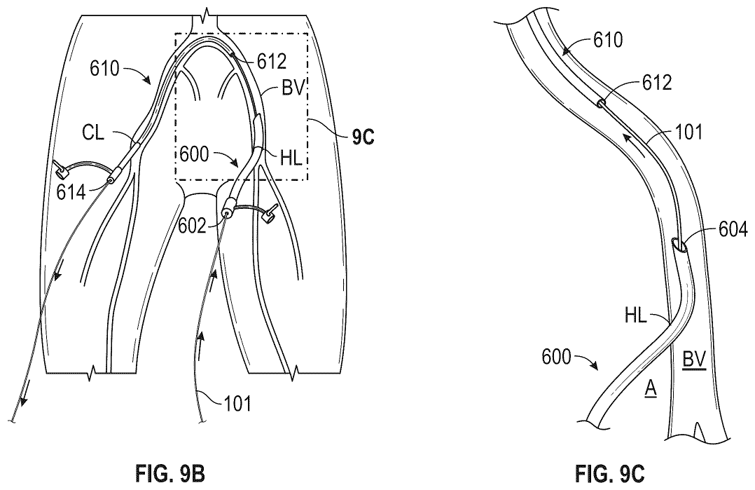

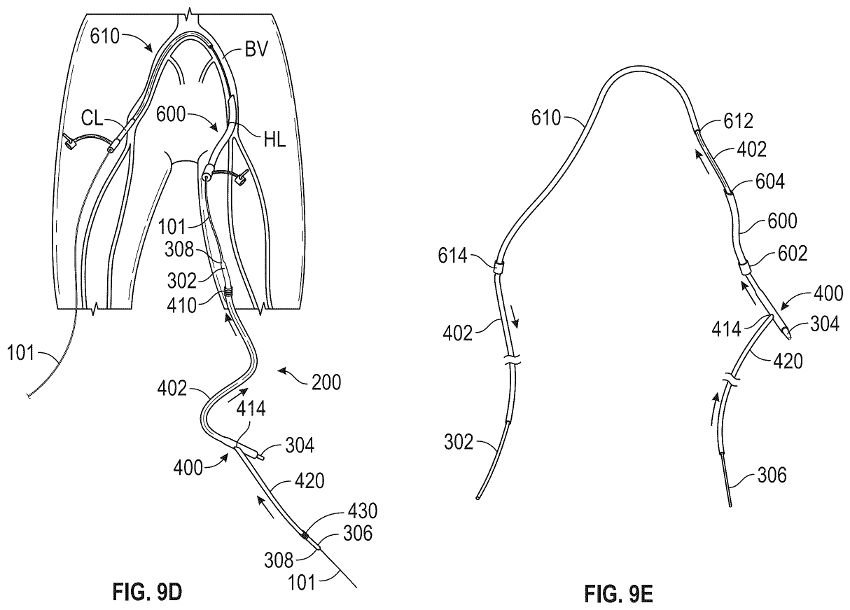

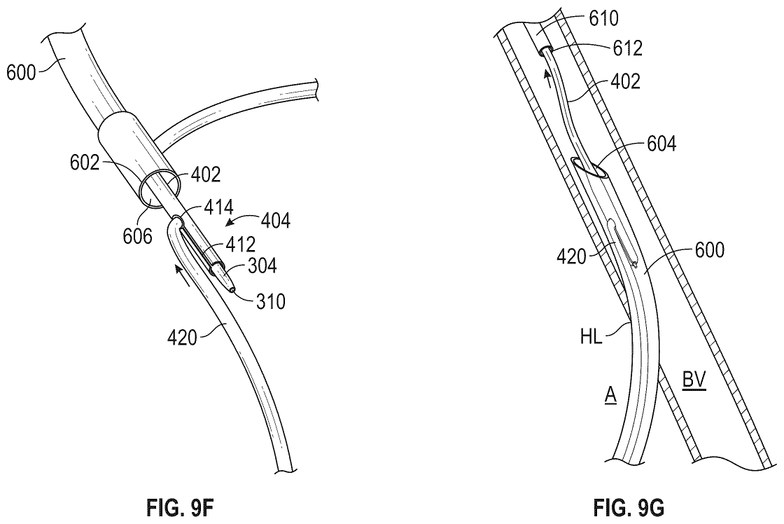

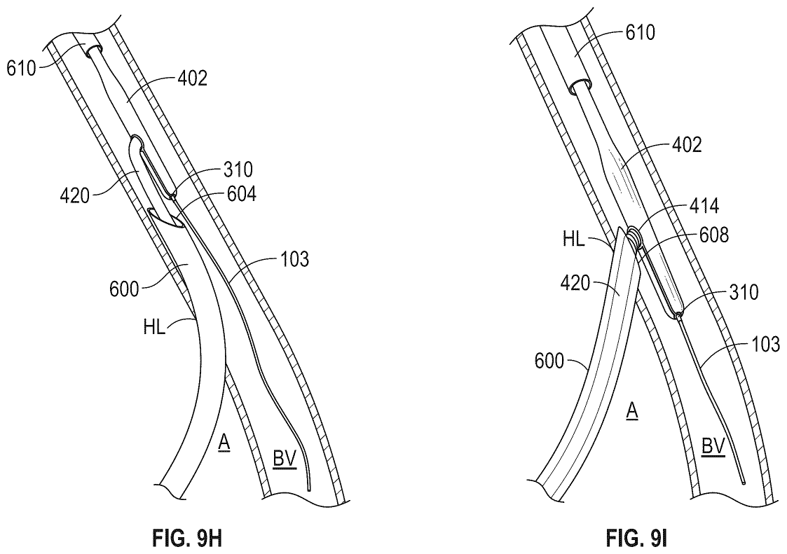

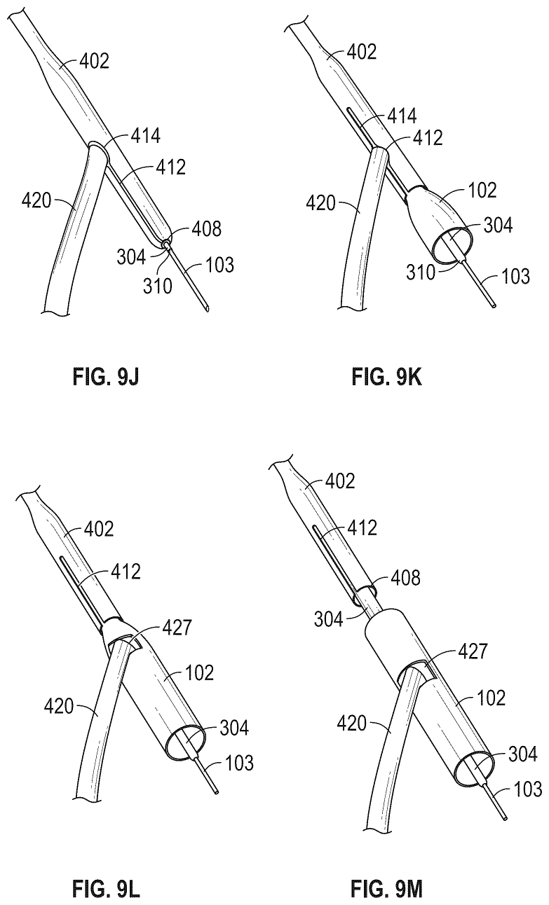

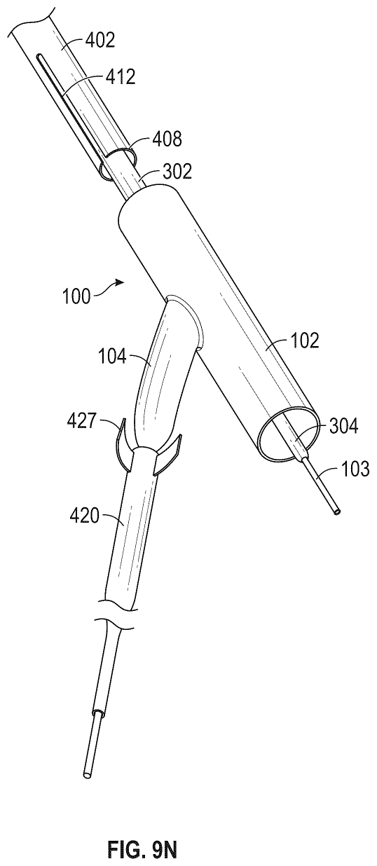

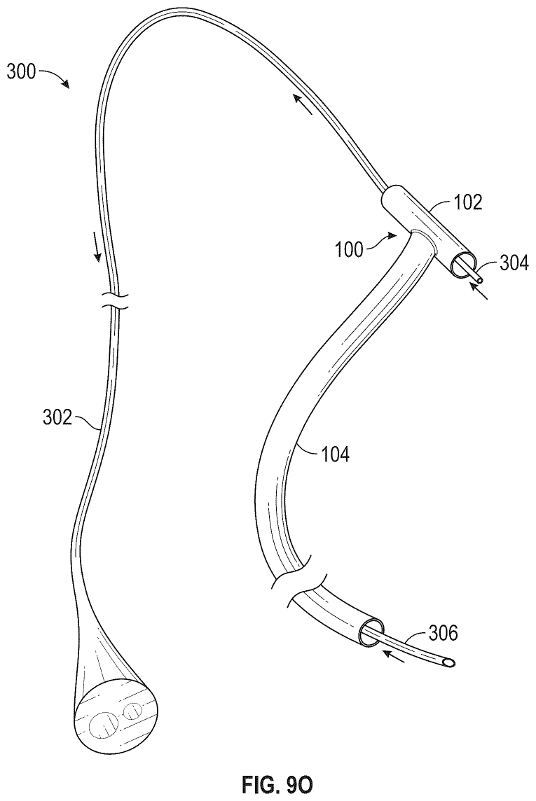

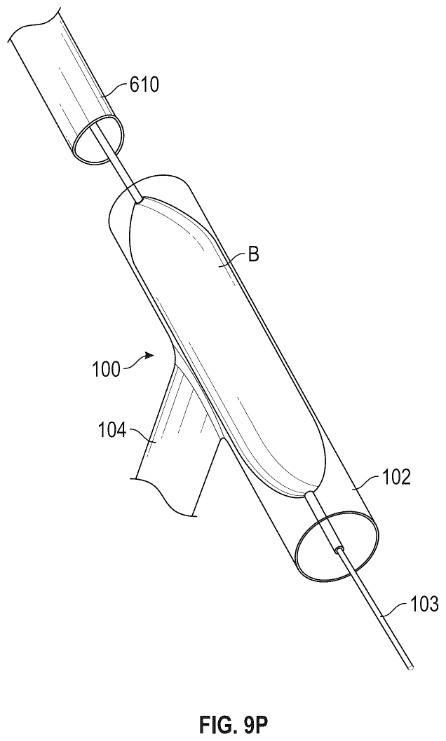

Referring now to FIG. 7, exemplary method 700 for delivering bifurcated stent 100 to a target vessel using delivery system 200 is provided. Some of the steps of method 700 may be further elaborated by referring to FIGS. 8A-9Q. Specifically, FIGS. 8A to 8D illustrate preparing delivery system 200 for delivery, and steps 702 to 714 of method 700 and FIGS. 9A to 9P describe delivering system 200 to a target blood vessel for implantation of bifurcated stent 100. To prepare bifurcated stent 100 for delivery, bifurcated stent 100 may be advanced over inner shaft 300, as shown in FIG. 8A. As shown in FIG. 8A, main portion 102 of bifurcated stent 100 may be disposed over at least a portion of distal main portion 302 and proximal main portion 304 of inner shaft 300, and side portion 104 of bifurcated stent 100 may be disposed over at least a portion of side portion 306 of inner shaft 300.

Bifurcated stent 100 may then be crimped from its expanded deployed state to its collapsed delivery state on inner shaft 300, as shown in FIG. 8B. As shown in FIG. 8C, second outer shaft 420 of outer shaft 400 may then be advanced over side portion 104 of bifurcated stent 100, such that side portion 104 is disposed within second outer shaft 420 in its collapsed delivery state, and flaps 427 of second outer shaft 420 engage at least a portion of main portion 102 of bifurcated stent 100 in its collapsed delivery state. Thus, flaps 427 may assist in maintaining main portion 102 of bifurcated stent 100 in its collapsed delivery state. First outer shaft 402 may then be advanced over distal main portion 302 of inner shaft 300 and the distal portion of main portion 102 of bifurcated stent 100 in its collapsed delivery state, followed by the proximal portion of main portion 102 in its collapsed delivery state, such that second outer shaft 420 and side portion 104 disposed therein slide along slot 412 until second outer shaft 420 is positioned within and extends from opening 414 of slot 412.

As shown in FIG. 8D, when first outer shaft 402 is disposed over main portion 102 (not shown), first outer shaft 402 is also disposed over flaps 427 of second outer shaft 420. Opening 414 may be sized and shaped to permit second outer shaft 420 to extend therethrough, while preventing flaps 427 from inadvertently being pulled through opening 414, to thereby secure second outer shaft 420 relative to first outer shaft 402. Accordingly, in the delivery configuration, main portion 102 of bifurcated stent 100 may be collapsed within first outer shaft 402 of outer shaft 400, e.g., within distal region 404 of first outer shaft 402, and side portion 104 may be collapsed within second outer shaft 420 such that side portion 104 extends through opening 414 of first outer shaft 402 within second outer shaft 420. FIG. 8E is a cross-sectional view of distal region 404 at proximal main portion 304. As shown in FIG. 8E, main portion 102 of bifurcated stent 100 may be disposed over proximal main portion 304 of inner shaft 300 having lumen 310 in its collapsed delivery state within first outer shaft 402 having slot 412.

At step 702, a first sheath, e.g., dedicated delivery sheath 600, may be inserted into the patient's vasculature at homolateral access point HL, e.g., through adjoining anastomosis A, and second sheath 610, e.g., a 6 Fr sheath, may be inserted into the patient's vasculature at a remote access point such as contralateral access point CL as shown in FIG. 9A. Alternatively, the remote access point may be a brachial access point, a radial access point, an axillary access point, a subclavian access point, a popliteal access point, a tibial access point, a pedal access point, etc., depending on the location of the anastomosis. As shown in FIG. 9A, sheath 600 may be inserted within the patient such that outlet 604 is disposed within the target location within blood vessel BV, adjacent to the carina forming anastomosis A, and inlet 602 is external to the patient via homolateral access point HL. Further, sheath 610 may be inserted within the patient such that outlet 612 of sheath 610 is disposed distal and adjacent to outlet 604 of sheath 600 within the target location within blood vessel BV, and inlet 614 is external to the patient via contralateral access point CL.

At step 704, guidewire 101, e.g., a 0.035″ guidewire, may be advanced through sheath 600 and sheath 610, as shown in FIGS. 9B and 9C. As shown in FIG. 9B, guidewire 101 may be inserted through inlet 602 of sheath 600, through homolateral access point HL via sheath 600, out of outlet 604 of sheath 600, through blood vessel BV and into outlet 612 of sheath 610 (as shown in FIG. 9C), through contralateral access point CL via sheath 610, and out of inlet 614 of sheath 610. Accordingly, guidewire 101 may span from outside of sheath 600 adjacent to homolateral access point HL to outside of sheath 610 adjacent to contralateral access point CL, as shown in FIG. 9B.

At step 706, delivery system 200, e.g., outer shaft 400 having bifurcated stent 100 disposed therein over inner shaft 300 in a collapsed delivery state, may be advanced handle-less over guidewire 101, as shown in FIGS. 9D to 9E. As shown in FIG. 9D, system 200 may be advanced over guidewire 101 via lumen 308 of inner shaft 300, such that guidewire 101 is inserted through lumen 308 of distal main portion 302 and side portion 306 of inner shaft 300. As shown in FIG. 9E, the distal ends of distal main portion 302 and first outer shaft 402 may be advanced handle-less over guidewire 101 through inlet 602 of sheath 600, through lumen 606 of sheath 600, out of outlet 604 of sheath 600, through blood vessel BV and into outlet 612 of sheath 610, through the lumen of sheath 610, and out of inlet 614 of sheath 610.

A handle, e.g., handle 500, may be removably coupled to each of the distal end of first outer shaft 402 outside of sheath 610 and the proximal end of second outer shaft 420 outside of sheath 600, to stabilize inner and outer shafts relative to handle 500, and accordingly to each other, and facilitate a safe manipulation, insertion and positioning of the bifurcated stent at the proper delivery site. For example, as described above, handle 500 may be advanced over distal main portion 302 of inner shaft 300 toward the distal end of first outer shaft 402 via lumen 516 of handle 500, then rotatable portion 504 may be rotated relative to first outer shaft 402 to mate threaded surface 410 of first outer shaft 402 with threaded surface 518 of handle 500, to thereby secure handle 500 to the distal end of first outer shaft 402. Moreover, as described above, distal main portion 302 may extend through engagement tube 524 in the unlocked state, and actuator 512 may be actuated to transition engagement tube 524 from the unlocked state to the locked state, e.g., by moving slider 526 relative to engagement tube 524, to thereby secure distal main portion 302 of inner shaft 300 relative to engagement tube 524 and handle 500. Another handle may similarly be removably coupled to the proximal end of second outer shaft 420 via threaded surface 430 of second outer shaft 420.

As shown in FIGS. 9F and 9G, second outer shaft 420 of outer shaft 400 may be folded against proximal region 404 of first outer shaft 402 as first outer shaft 402 and second outer shaft 420 are advanced together through inlet 602 and within lumen 606 of sheath 600 toward outlet 604 of sheath 600, until first outer shaft 402 is completely disposed outside of sheath 600 within blood vessel BV, as shown in FIG. 9H.

At step 708, guidewire 103, e.g., a 0.018″ guidewire, may be inserted through lumen 310 of inner shaft 300, as further shown in FIG. 9H. For example, guidewire 103 may be inserted through lumen 310 at the distal end of distal main portion 302 and out of the proximal end of proximal main portion 304, such that at least a portion of guidewire 103 is disposed within blood vessel BV, to facilitate alignment and deployment of bifurcated stent 100 within blood vessel BV and anastomosis A, e.g., by maintaining the direction for proper stent deployment. Accordingly, guidewire 103 may span from outside of sheath 610 adjacent to contralateral access point CL to within blood vessel BV adjacent to anastomosis A.

At step 710, main portion 102 of bifurcated stent 100 disposed within first outer shaft 402 in its collapsed delivery state may be positioned within blood vessel BV, and side portion 104 of bifurcated stent 100 disposed within second outer shaft 420 in its collapsed delivery state may be positioned within anastomosis A, as shown in FIG. 9I. For example, system 200 may be pushed and pulled via the distal ends of distal main portion 302 of inner shaft 300 and/or first outer shaft 402, and/or the proximal ends of side portion 306 of inner shaft 300 and/or second outer shaft 420, until bifurcated stent 100 is in the desired position within blood vessel BV and anastomosis A, as shown in FIG. 9I, which may be visually observed by observing the relative position between marker 608 of sheath 600 and bifurcated stent 100 via, e.g., fluoroscopy. As described above, by having both inner shaft 300 and outer shaft 400 fixed relative to each other via handle 500 during maneuvering and positioning of bifurcated stent 100 at the target location, the safe and accurate deployment of bifurcated stent 100 via inner shaft 300 and outer shaft 400 may be ensured. Moreover, flaps 427 secured over main portion 102 within first outer shaft 402 may facilitate maneuvering of system 200 by stabilizing second outer shaft 420 relative to first outer shaft 402.

At step 712, first outer shaft 402 may be retracted distally relative to bifurcated stent 100 through second sheath 610 to deploy main portion 102 of bifurcated stent 100 within the target blood vessel, and second outer shaft 420 may be retracted proximally relative to bifurcated stent 100 through first sheath 600 to deploy side portion 104 of bifurcated stent 100 within the adjoining anastomosis. Specifically, as shown in FIGS. 9J to 9M, first outer shaft 402 may be retracted distally relative to bifurcated stent 100, e.g., via handle 500 coupled to the distal end of first outer shaft 402, through sheath 610, such that main portion 102 of bifurcated stent 100 is exposed beyond the proximal end of first outer shaft 402 and transitions from its collapsed delivery state to its expanded deployed state within the target blood vessel. For example, as described above, actuator 508 of handle 500 may be actuated, e.g., via a rack and pinion system, to incrementally retract first outer shaft 402 relative to inner shaft 300 until main portion 102 is at least partially or fully deployed within the target blood vessel.

As shown in FIGS. 9J and 9K, as first outer shaft 402 is retracted distally relative to bifurcated stent 100, side portion 104 disposed within second outer shaft 420 in its collapsed delivery state is guided along slot 412 from opening 414 toward the proximal end of first outer shaft 402 until side portion 104 is disengaged from first outer shaft 402, as shown in FIGS. 9L and 9M. First outer shaft 402 may further be retracted distally through sheath 610 over distal main portion 302 of inner shaft 300 to remove first outer shaft 402 from the patient's body. For example, distal main portion 302 may be decoupled from handle 500 as described above, such that handle 500 and first outer shaft 402 coupled thereto may be retracted and removed over distal main portion 302 of inner shaft 300. As described above, when second outer shaft 420 includes flap 427, such that flap 427 is sandwiched between first outer shaft 402 and side portion 104 in its collapsed delivery state, first outer shaft 402 must be removed from main portion 102 to thereby expose flap 427 prior to retraction of second outer shaft 420 relative to bifurcated stent 100 to deploy side portion 104.

Next, as shown in FIG. 9N, second outer shaft 420 may be retracted proximally relative to bifurcated stent 100, e.g., via a handle coupled to the proximal end of second outer shaft 420, through sheath 600, such that side portion 104 of bifurcated stent 100 is exposed beyond the distal end of second outer shaft 420 and transitions from its collapsed delivery state to its expanded deployed state within the anastomosis, to thereby implant bifurcated stent 100 within the target blood vessel and adjoining anastomosis. Second outer shaft 420 may further be retracted proximally through sheath 600 over side portion 306 of inner shaft 300 to remove second outer shaft 420 from the patient's body. For example, side portion 306 may be decoupled from handle 500 as described above, such that handle 500 and second outer shaft 420 coupled thereto may be retracted and removed over side portion 306 of inner shaft 300. At step 714, inner shaft 300 may be retracted distally relative to deployed bifurcated stent 100 through sheath 610 to remove inner shaft 300 from the patient's body, as shown in FIG. 9O. In some embodiments, a balloon catheter having balloon B may be positioned within one or both of main portion 102 or side portion 104 of bifurcated stent 100, and inflated to apply a radial force to the respective portions of bifurcated stent 100 to facilitate further deployment of bifurcated stent 100, if necessary, as shown in FIG. 9P.

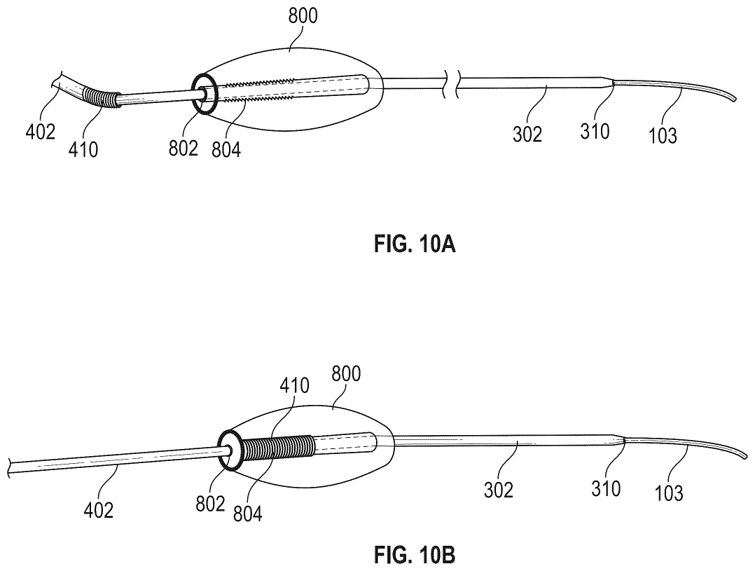

Referring now to FIGS. 10A and 10B, an exemplary alternative removable handle is provided. Handle 800 may be configured to be held and controlled by a user, and may have lumen 802 extending therethrough. Like lumen 516 of handle 500, lumen 802 of handle 800 may be sized and shaped to receive distal main portion 302 of inner shaft 300 and/or side portion 306 of inner shaft 300 therethrough. Moreover, at least a portion of lumen 802 extending through handle 800 may include threaded inner surface 804 configured to removably mate with threaded surface 410 of first outer shaft 402 and/or threaded surface 430 of second outer shaft 420. Accordingly, at least a portion of lumen 802 extending through handle 800 may be sized and shaped to receive the threaded surfaces of first outer shaft 402 and/or second outer shaft 420. For example, handle 800 may be advanced over distal main portion 302 of inner shaft 300, as shown in FIG. 10A until lumen 802 comes into contact with threaded surface 410 of first outer shaft 402. Handle 800 may then be rotated relative to first outer shaft 402 to receive threaded surface 410 within lumen 802 to thereby releasably secure handle 800 to outer shaft 400. Handle 800 and outer shaft 400 coupled thereto may then be pulled proximally relative to inner shaft 300 by the user to translationally move outer shaft 400 relative to inner shaft 300, e.g., to thereby retract/remove outer shaft 400 from bifurcated stent 100 while inner shaft 300 remains stationary relative to the target blood vessel and adjoining anastomosis. Accordingly, the removable handles described herein may permit delivery of a large caliber stent from a remote location from the implantation site while only having to accommodate for the diameter of the delivery shafts, e.g., the outer shafts. As will be understood by a person having ordinary skill in the art, system 200 may include two separate handles, e.g., handle 800 and/or handle 500, for removably coupling to each of first outer shaft 402 and second outer shaft 420 during delivery of bifurcated stent 100.

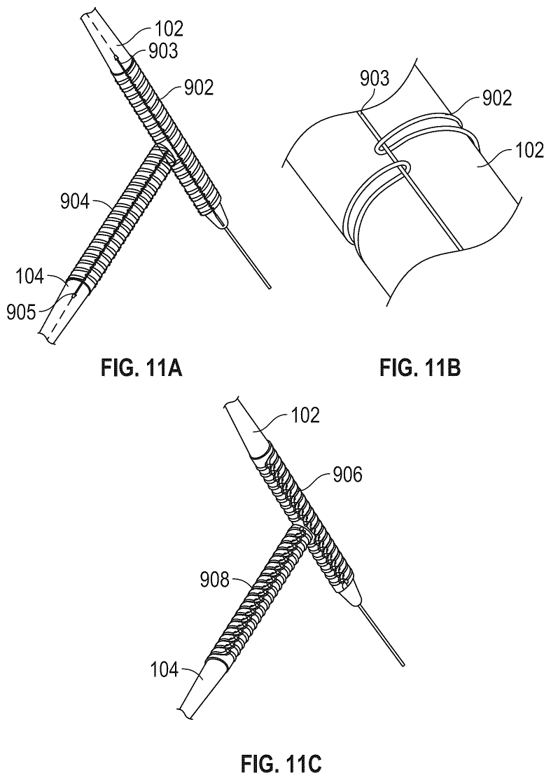

Referring now to FIGS. 11A and 11B, an alternative exemplary outer shaft assembly is provided. Specifically, instead of outer shaft 400, e.g., first outer shaft 402 and second outer shaft 420, system 200 may include a pair of soft continuous wires, e.g., first wire shaft 902 and second wire shaft 904, configured to wrap around and maintain bifurcated stent 100 in its crimped, collapsed delivery state. For example, first wire shaft 902 may be wrapped around main portion 102 of bifurcated stent 100 in its collapsed delivery state, in a predefined pattern about first stylet 903, such that first stylet 903 maintains first wire shaft 902 in position relative to main portion 102. The distal end of first stylet 903 may extend outside of the patient at the remote access point, such that, upon removal of first stylet 903, first wire shaft 902 unravels and disengages with main portion 102, thereby permitting main portion 102 to self-expand within the target blood vessel. Accordingly, first wire shaft 902 may be coupled to first stylet 903 in a manner such that, upon removal of first stylet 903 and upon unraveling of first wire shaft 902, first wire shaft 902 may be removed along with first stylet 903.

Similarly, second wire shaft 904 may be wrapped around side portion 104 of bifurcated stent 100 in its collapsed delivery state, in a predefined pattern about second stylet 905, such that second stylet 905 maintains second wire shaft 904 in position relative to side portion 104. The proximal end of second stylet 905 may extend outside of the patient at the homolateral access point, such that, upon removal of second stylet 905, second wire shaft 904 unravels and disengages with side portion 104, thereby permitting side portion 104 to self-expand within the adjoining anastomosis. Accordingly, second wire shaft 904 may be coupled to second stylet 905 in a manner such that, upon removal of second stylet 905 and upon unraveling of second wire shaft 904, second wire shaft 904 may be removed along with second stylet 905.

Alternatively, as shown in FIG. 11C, system 200 may include a pair of soft continuous wires, e.g., first wire shaft 906 and second wire shaft 908, configured to wrap around and maintain main portion 102 and side portion 104 of bifurcated stent 100, respectively, in its crimped, collapsed delivery state. For example, first wire shaft 906 may be wrapped around main portion 102 of bifurcated stent 100 in its collapsed delivery state, in a predefined Viabahn pattern, and second wire shaft 908 may be wrapped around side portion 104 of bifurcated stent 100 in its collapsed delivery state, in a predefined Viabahn pattern. The distal free end of first wire shaft 906 at the remote access point and the proximal free end of second wire shaft 908 at the homolateral access point may be pulled relative to bifurcated stent 100 to thereby cause first wire shaft 906 and second wire shaft 908, respectively, to unravel and disengage from main portion 102 and side portion 104, respectively, thereby permitting self-expansion of the respective portions of bifurcated stent 100 at the target location.

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed:

1. A system for implanting a bifurcated stent comprising a main portion and a side portion within a blood vessel and an adjoining anastomosis, the system comprising:
an inner shaft comprising a first portion configured to support the main portion of the bifurcated stent and a second portion configured to support the side portion of the bifurcated stent;
a first outer shaft comprising a lumen sized and shaped to receive the main portion of the bifurcated stent in a collapsed delivery state over the first portion of the inner shaft, and a slot extending longitudinally from a proximal end of the first outer shaft, the slot sized and shaped to accommodate the side portion of the bifurcated stent; and
a second outer shaft comprising a lumen sized and shaped to receive the side portion of the bifurcated stent in a collapsed delivery state over the second portion of the inner shaft,
wherein the first outer shaft is configured to be retracted distally relative to the bifurcated stent to transition the main portion of the bifurcated stent from its collapsed delivery state to an expanded deployed state within the blood vessel, and
wherein the second outer shaft is configured to be retracted proximally relative to the bifurcated stent to transition the side portion of the bifurcated stent from its collapsed delivery state to an expanded deployed state within the anastomosis.

2. The system of claim 1, wherein the second portion of the inner shaft extends from a side of the first portion of the inner shaft.

3. The system of claim 1, wherein the inner shaft comprises a guidewire lumen extending entirely through the first portion of the inner shaft.

4. The system of claim 1, wherein the inner shaft comprises a guidewire lumen extending through at least a portion of the first portion of the inner shaft and entirely through the second portion of the inner shaft.

5. The system of claim 1, wherein the inner shaft comprises a stiffness configured to facilitate alignment of the main portion of the bifurcated stent within the blood vessel and the side portion of the bifurcated stent within the anastomosis.

6. The system of claim 1, wherein the first and second outer shafts are sized and shaped for advancement through the anastomosis to the blood vessel.

7. The system of claim 1, wherein a distal end of the second outer shaft comprises one or more flaps configured to engage with the main portion of the bifurcated stent within the first outer shaft.

8. The system of claim 7, wherein the slot of the first outer shaft is sized and shaped to permit the second outer shaft to extend therethrough while preventing the one or more flaps from being pulled through the slot when the one or more flaps are disposed over the main portion of the bifurcated stent within the first outer shaft.

9. The system of claim 1, further comprising a handle configured to be removably coupled to at least one of a distal region of the first outer shaft or a proximal region of the second outer shaft.

10. The system of claim 9, wherein the handle comprises a lumen having a first threaded surface, and wherein at least one of the distal region of the first outer shaft or the proximal region of the second outer shaft comprises a corresponding threaded surface configured to be removably coupled to the first threaded surface.

11. The system of claim 10, wherein the handle further comprises an engagement tube having a lumen sized and shaped to receive at least one of the first portion of the inner shaft or the second portion of the inner shaft, the engagement tube configured to be actuated to transition between an unlocked state where the lumen slidably receives the at least one of the first portion of the inner shaft or the second portion of the inner shaft and a locked state where the lumen engages and secures the at least one of the first portion of the inner shaft or the second portion of the inner shaft to the engagement tube.

12. The system of claim 11, wherein the engagement tube comprises a pair of locking portions, and wherein the handle further comprises a slider having a tapered portion defining a slot extending within the slider, the slider configured to be actuated to transitionally move along the pair of locking portions between an unlocked position where the slider is not engaged with the pair of locking portions and a locked position where the pair of locking portions is received within the slot and moved toward each other via the tapered portion to decrease an area of the lumen of the engagement tube to thereby secure the at least one of the first portion of the inner shaft or the second portion of the inner shaft to the engagement tube.

13. The system of claim 11, wherein the handle comprises an actuator configured to be actuated to translationally move the at least one of the distal region of the first outer shaft or the proximal region of the second outer shaft relative to the at least one of the first portion of the inner shaft or the second portion of the inner shaft, respectively, when the at least one of the distal region of the first outer shaft or the proximal region of the second outer shaft is removably coupled to the handle and the at least one of the first portion of the inner shaft or the second portion of the inner shaft is secured to the engagement tube.

14. The system of claim 13, wherein the actuator comprises a pinion, and wherein the handle further comprises a rack configured to moveably engage with the pinion to translationally move the at least one of the distal region of the first outer shaft or the proximal region of the second outer shaft relative to the at least one of the first portion of the inner shaft or the second portion of the inner shaft, respectively.

15. The system of claim 1, wherein, when the main portion of the bifurcated stent is disposed within the lumen of the first outer shaft in its collapsed delivery state, the side portion of the bifurcated stent extends through the slot of the first outer shaft.

16. The system of claim 15, wherein, as the first outer shaft is retracted distally relative to the bifurcated stent, the side portion of the bifurcated stent is guided along the slot of the first outer shaft.

17. The system of claim 1, further comprising the bifurcated stent comprising the main portion and the side portion, the bifurcated stent configured to transition between a collapsed delivery state and an expanded deployed state.

18. The system of claim 17, wherein a portion of the bifurcated stent adjacent to where the main portion is coupled to the side portion comprises a stiffness greater than other portions of the bifurcated stent.

19. The system of claim 1, further comprising:
a first sheath sized and shaped for advancement through the anastomosis to the blood vessel, the first sheath comprising a lumen sized and shaped to receive the first and second outer shafts therethrough,
wherein a distal end of the sheath comprises a tapered profile.

20. The system of claim 19, wherein the first sheath is sized and shaped for advancement through the anastomosis to the blood vessel via a homolateral access point of the blood vessel.

21. The system of claim 19, further comprising a second sheath sized and shaped for advancement through the blood vessel via a remote access point of the blood vessel, the second sheath comprising a lumen sized and shaped to receive the first outer shaft therethrough.

22. A method for implanting a bifurcated stent comprising a main portion and a side portion within a blood vessel and an adjoining anastomosis, the method comprising:
inserting a first sheath within the blood vessel via a homolateral access point;
inserting a second sheath within the blood vessel via a remote access point;
inserting a guidewire through the first sheath and the second sheath;
advancing an inner shaft over the guidewire through the anastomosis to the blood vessel, the inner shaft supporting the bifurcated stent in a collapsed delivery state, the main portion of the bifurcated stent disposed within a first outer shaft in the collapsed delivery state and the side portion of the bifurcated stent disposed within a second outer shaft in the collapsed delivery state, the first outer shaft comprising a longitudinally extending slot sized and shaped to accommodate the side portion of the bifurcated stent;
positioning the first outer shaft within the blood vessel and the second outer shaft within the anastomosis;
retracting the first outer shaft distally relative to the bifurcated stent via the remote access point to transition the main portion of the bifurcated stent from the collapsed delivery state to an expanded deployed state within the blood vessel, such that the side portion of the bifurcated stent is guided along the slot of the first outer shaft; and
retracting the second outer shaft proximally relative to the bifurcated stent via the homolateral access point to transition the side portion of the bifurcated stent from its collapsed delivery state to an expanded deployed state within the anastomosis.

23. The method of claim 22, wherein a first portion of the inner shaft supports the main portion of the bifurcated stent in the collapsed delivery state within the first outer shaft, and wherein a second portion of the inner shaft extending from the first portion of the inner shaft supports the side portion of the bifurcated stent in the collapsed delivery state within the second outer shaft.

24. The method of claim 23, wherein advancing the inner shaft over the guidewire comprises advancing the inner shaft over the guidewire via a guidewire lumen extending through at least a portion of the first portion of the inner shaft and through the second portion of the inner shaft.

25. The method of claim 24, further comprising inserting a second guidewire through a second guidewire lumen extending entirely through the first portion of the inner shaft, such that the second guidewire is disposed through the remote access point within the blood vessel to facilitate deployment of the bifurcated stent.

26. The method of claim 22, wherein advancing the inner shaft over the guidewire comprises advancing the inner shaft and the first outer shaft through the first sheath and the second sheath such that distal ends of the inner shaft and the first outer shaft extend through the remote access point.

27. The method of claim 26, further comprising:
coupling a handle to the distal end of the first outer shaft,
wherein retracting the first outer shaft distally relative to the bifurcated stent comprises retracting the first outer shaft via the handle.

28. The method of claim 22, further comprising:
coupling a handle to a proximal end of the second outer shaft,
wherein retracting the second outer shaft proximally relative to the bifurcated stent comprises retracting the second outer shaft via the handle.

29. The method of claim 22, wherein positioning the first outer shaft within the blood vessel and the second outer shaft within the anastomosis comprises visualizing a distal end of the first sheath via markers disposed on the distal end of the first sheath.

30. The method of claim 22, further comprising retracting the inner shaft distally relative to the bifurcated stent via the remote access point to remove the inner shaft from the blood vessel.
